(19) United States
(12) Patent Application Publication
(10) Pub. No.: US 2010/0204589 A1
(43) Pub. Date: Aug. 12, 2010

(54) NON-INVASIVE INTRACRANIAL PRESSURE SENSOR

(75) Inventors: Marek Swoboda, Philadelphia, PA (US); Matias G. Hochman, Philadelphia, PA (US); Frederick J. Fritz, Skillman, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: NEURODX DEVELOPMENT LLC, Philadelphia, PA (US)

(21) Appl. No.: 12/671,468

(22) PCT Filed: Aug. 1, 2008
(86) PCT No.: PCT/US08/71888

§ 371 Date: Jan. 29, 2010

(30) Foreign Application Priority Data
Aug. 2, 2007 (US) 60953606
Jun. 6, 2008 (US) 61059496

(51) Int. Cl. A61B 5/021

(52) U.S. Cl. 600/485

(57) ABSTRACT

A system and method for non-invasively detecting intracranial pressure (ICP) of a living being by detecting impedance mismatches between carotid arteries and cerebral vessels via a reflection of the carotid pressure waveform using a pressure sensor positioned against the palpable carotid artery, as well as analyzing the reflection and comparing the analysis with known cerebral vasculature data, to calculate ICP non-invasively. A remote blood pressure waveform can also be used to compensate for blood system impedance.

### CROSS-REFERENCE TO RELATED APPLICATIONS

This PCT application claims the benefit under 35 U.S.C. §119(e) of Provisional Application Ser. Nos. 60/953,606 filed on Aug. 2, 2007, entitled NON-INVASIVE PULSE WAVEFORM ANALYSIS FOR MEASURING INTRACRANIAL PRESSURE IN TRAUMATIC BRAIN INJURY and 61/059,496 filed on Jun. 6, 2008, entitled NONINVASIVE INTRACRANIAL PRESSURE MONITOR, and all of whose entire disclosures are incorporated by reference herein.

### BACKGROUND OF THE INVENTION

1. Field of Invention

The present invention generally relates to medical devices and more particularly to systems and methods for measuring intracranial pressure non-invasively.

2. Description of Related Art

Intracranial pressure (ICP) monitoring is a critical unmet need in the neurosurgical market. Current ICP measurement techniques require placement of a pressure probe in contact with cerebrospinal fluid (CSF). These techniques carry inherent surgical risks, require specialized facilities, and suffer from data quality limitations (such as measurement drift) resulting from the reactive biological interface. The most common conditions that cause increased ICP and that may require monitoring are serious head injuries (approximately 9,400 non-military cases in the U.S. annually), brain tumors (approximately 51,000 cases), CSF shunting (approximately 41,000 cases), and pseudotumor cerebri (approximately 11,000 cases). In addition, brain aneurysms and hemorrhagic strokes may require monitoring of ICP.

The importance of monitoring ICP in neurosurgical and neurological patients, and the limitations with current methods (invasiveness, high infection rates, limited precision, high failure rates) are well known and have recently been reviewed (Ref. 1; reference citations are located at the end of the instant Specification). Unlike most organs, pressure within the brain is not coupled to atmospheric pressure, as it is surrounded by a stiff skull 2 (FIG. 1B). ICP is contributed to by the volume of CSF within the brain ventricles and surrounding the brain, and by the cerebral blood flow that contributes to CSF formation (Refs. 2-4). In particular, there are four carotid arteries: an external and an internal carotid on each side of the body (see FIG. 1A) that supply blood to the head and brain. The blood is filtered at the choroid plexus 9 (see FIG. 1B) to form CSF 7 which accumulates in the internal brain ventricles, and in the subarachnoid space (see dura 8) around the brain 1. Any factor that disturbs the normal pressure dynamics within the intracranial compartment (trauma and swelling, space occupying lesion, obstruction of fluid drainage pathways) can lead to elevated ICP. An increase in ICP causes compression of the brain tissues, starting with the ventricular and vascular spaces, and impedance of cerebral blood flow, leading to ischemia and brain damage (Ref. 5).

The CDC estimates that 1.4 million American civilians suffer from traumatic brain injury annually. Approximately, 1.1 million are treated in the emergency room and released. Those patients would be tested one time for elevated ICP. Ongoing ICP monitoring or repeat testing is conducted on the approximately 235,000 traumatic brain injury patients who are hospitalized annually, as well as in brain tumor patients (approximately 51,000 cases), CSF shunt patients (approximately 41,000), and pseudotumor cerebri patients (approximately 11,000). In addition, brain aneurysms and hemorrhagic strokes may require monitoring of ICP. Patients with shunts, tumors, and pseudotumor cerebri would also benefit from serial, ambulatory monitoring after they are released from the hospital.

It is estimated that at least 20% of all military casualties, and as many as 60% of those in today's combat zones, include traumatic head and brain injuries. Since 2001, in Afghanistan alone, approximately 2,100 troops have been diagnosed with TBI, although it is estimated that up to 150,000 troops may have suffered mild TBI (concussion) from roadside bombs, and it is increasingly recognized that these “missed” TBI's can manifest months or even years later. In addition to the severity and location of the injury, the symptoms of, and prognosis for, traumatic brain injury patients depends on the speed with which the injury can be properly assessed and treated. The proposed device will therefore meet an immediate need for portable and noninvasive devices for early management of head injuries of military personnel in the field.

Measuring ICP is an essential component in the management of neurosurgical conditions, and is integrated into diagnosis, prognosis, and monitoring response to treatment. Prompt detection and treatment of cerebral hypertension can eliminate potential secondary insults before they cause severe injury to the brain. For acute neuropathological states including head trauma, CSF shunt blockage, and hematoma, ICP can be critical in determining appropriate treatment modalities (Refs. 6-7). The association between the severity of intracranial hypertension and poor outcome following head injury is well recognized (Refs. 8-9). Following head trauma, the likelihood of mortality is substantially lowered when ICP is routinely monitored and controlled (Refs. 10-11). For chronic neurosurgical conditions, including tumor and hydrocephalus, ongoing monitoring enables assessment of response to treatment (Refs. 12-14). The importance of ICP measurement is also increasingly being recognized in the management of encephalitis (Ref. 15) and stroke (Ref. 16). It is probable that patient and time-dependent differences in ICP exist, making it difficult to define a universally “normal” ICP value—suggests the importance of ongoing measurements to evaluate changes and trends.

As shown in FIG. 2, the gold standard for ICP measurement involves drilling a hole 3 into the skull 2 and placing a catheter 4 within the ventricles of the brain 1 from where fluid pressure is measured directly (Ref. 17). Other methods include inserting a fiber optic probe within the brain parenchyma (Ref. 18), inserting a metal bolt into the subarachnoid layer of the brain 1 (Ref. 19), or placing a probe in the epidural space between the inner surface of the skull 2 and the superficial layer of the brain (Ref. 20). These methods are all invasive, and carry risks of hemorrhage, infection, and obstruction. Furthermore direct contact between the probe and reactive biological tissues commonly results in sensor drift and malfunction. These invasive methods of measuring ICP can only be performed in specialized facilities where neurosurgeons are available. Previous attempts to measure intracranial pressure using intraocular tonometry (Ref. 21), MRI scanning algorithms (Ref. 22), acoustic emissions (Ref. 23), visual evoked potentials (Ref. 24), transcranial Doppler (Ref. 25), bioimpedance (Ref. 26), ultrasonic resonance (Ref. 27), skull pulsation (Ref. 28) and other noninvasive techniques have been hampered by expensive, cumbersome and non-portable equipment, as well as complex and unreliable software. All of these techniques require specialized training and most have failed validation trials. None has gained clinical acceptance. A device that can rapidly and accurately determine ICP non-invasively, that is portable, and that can be operated by first responders and general health practitioners would greatly improve the incorporation of ICP measurement as a routine modality for a variety of neurosurgical applications.

Thus, there remains a need for a device that overcomes these limitations, enabling rapid, non-invasive measurement of ICP in a serial ambulatory setting (such as by first responders, in the military field, or following discharge from hospital).

All references cited herein are incorporated herein by reference in their entireties.

### BRIEF SUMMARY OF THE INVENTION

A system for measuring intracranial pressure (ICP) of a living being non-invasively, wherein the system comprises: a sensor (e.g., a piezoresistive transducer) for detecting blood pressure (e.g., carotid artery blood pressure waveform) non-invasively; an analyzer that receives the blood pressure information and derives at least one parameter that correlates with ICP (e.g., a time delay between systolic maximum and the dicrotic notch) to provide ICP data from the blood pressure information; and an output device (e.g., a monitor) for displaying the ICP data.

A method for measuring intracranial pressure (ICP) of a living being non-invasively wherein the method comprises: non-invasively detecting blood pressure (e.g., carotid artery blood pressure waveform) of the living being; analyzing a feature of that detected blood pressure that correlates with ICP (e.g., a time delay between systolic maximum and the dicrotic notch) to provide ICP data from the feature of the detected blood pressure; calculating ICP from the feature of the detected blood pressure.

### BRIEF DESCRIPTION OF SEVERAL VIEWS OF THE DRAWINGS

The invention will be described in conjunction with the following drawings in which like reference numerals designate like elements and wherein:

FIG. 1A is a diagrammatic view of a human head/neck showing two of the four carotid arteries (an external and an internal carotid on each side of the body) that supply blood to the head and brain;

FIG. 1B is a diagrammatic view of a human brain and shows that the blood is filtered at the choroid plexus to form cerebrospinal fluid (CSF) which accumulates in the internal brain ventricles, and in the subarachnoid space around the brain;

FIG. 2 is the current “gold standard” for measuring intracranial pressure involves passing a catheter with a pressure sensing device through a hole in the skull, and inserting a pressure-sensing device in the internal ventricles of the brain;

FIG. 2A is a block diagram of the present invention;

FIG. 2B is a flow diagram of the method of the present invention;

FIG. 2C depicts a step in the flow diagram of FIG. 2B that correlates with changing ICP;

FIG. 3 shows the relationship between volume and pressure which can be predicted from the intracranial pressure volume curve; the relationship between volume and pressure is different at lower (A) vs. higher (B) intracranial volumes;

FIG. 4 is a cross-sectional view of a primary pressure sensor of the present invention for collecting carotid artery pressure waveform data, as well as a reference sensor for collecting reference artery pressure waveform data;

FIG. 4A is an isometric view of the pressure transducer of FIG. 4;

FIG. 4B depicts a flow diagram for data collection, conditioning and analysis utilized in the analyzer of the present invention;

FIG. 5A depicts “averaging” of pulse waveforms collected over time to produce a “typical” representative waveform for feature mining;

FIG. 5B depicts the relationship between leg elevation and one feature of the typical carotid artery blood pressure waveform (CABPW): the time between systolic maximum and the dicrotic notch (parameter X3);

FIG. 6 depicts an animal model for controlling and measuring ICP where a double-bored needle is inserted into the cisterna magna of an anesthetized animal stabilized within a sterotaxic head holder; one branch of the needle is attached to a pressure transducer for direct measurement of cisternal ICP, while the other is attached to a reservoir bottle. ICP measurements derived from carotid waveform measurements can be correlated to direct ICP measurements made via the cisternal needle;

FIGS. 7A-7E depict pressure pulse waveform derivatives; and

FIG. 8 is a phase plane plot of the carotid artery blood pressure waveform (CABPW) versus its first derivative, referred to as X3.

### DETAILED DESCRIPTION OF THE INVENTION

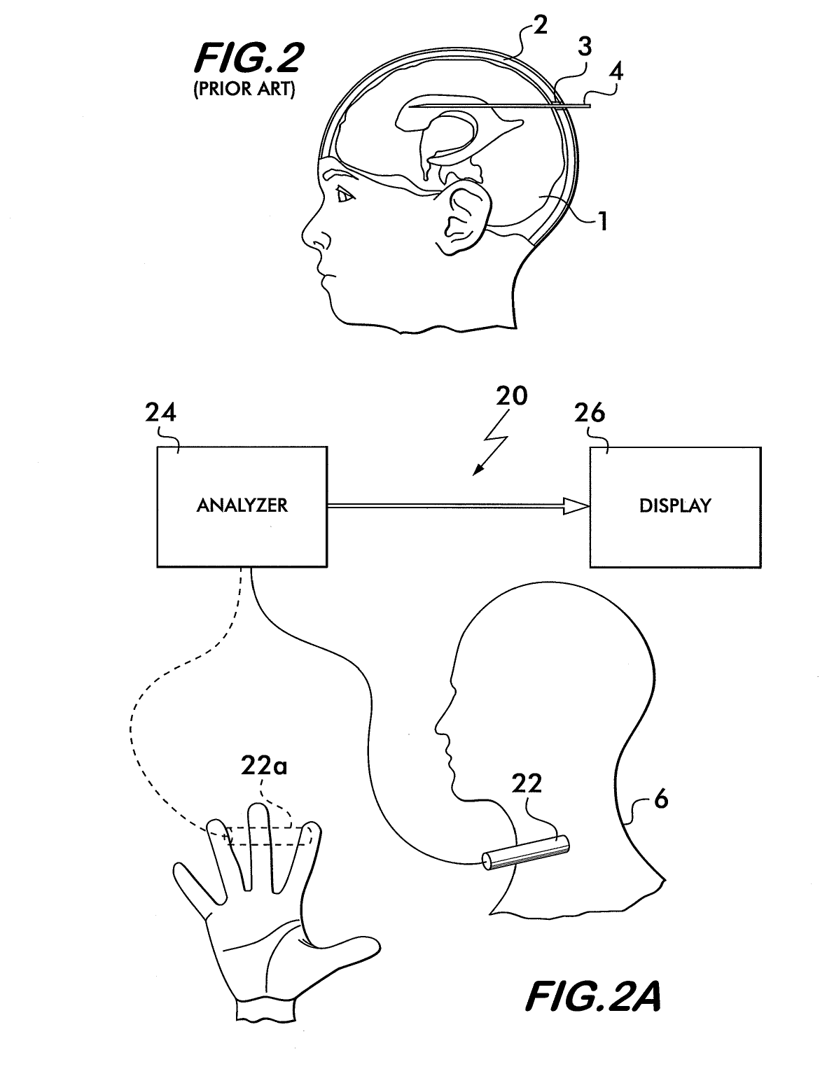

The present invention 20 is a non-invasive, hand-held device for measuring intracranial pressure (ICP). FIG. 2A depicts a block diagram of the system 20 which comprises a primary sensor 22, an analyzer 24 and an output device 26 (e.g., a monitor) for displaying the ICP and associated data. A reference sensor 22a (as will be discussed in detail later) may also be used but is not required. FIGS. 2B and 2C provide a flow diagram of the method 100 of the present invention.

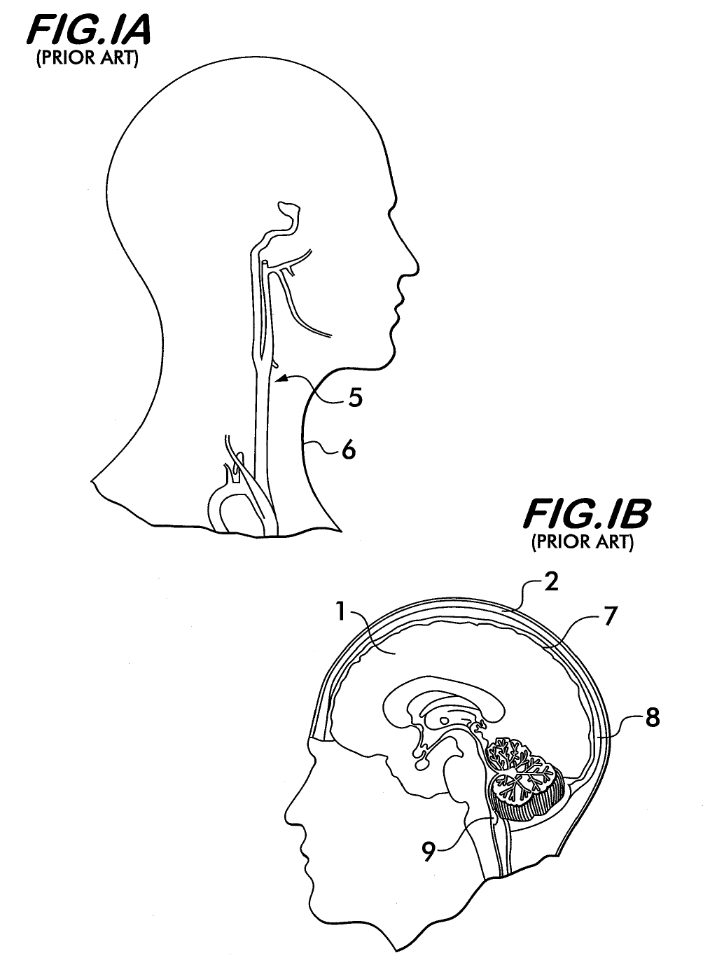

The invention 20 derives ICP from quantitative analysis of the pulse pressure waveform in the arteries supplying blood to the brain and preferably also based upon reference arteries (e.g., artery in the index finger). As explained previously, blood reaches the brain (mainly) via branches of the common carotid arteries 5 (see FIG. 1A). The carotid pressure wave is partly reflected upon striking the smaller diameter (and higher hydraulic impedance) cerebral vascular bed and this reflection contributes to the complex overall shape of the carotid pressure waveform. The impedance mismatch, and resulting wave reflection, is dependent on ICP which limits cerebral vascular compliance by compression. Because the carotid arteries are relatively superficial in the neck 6 (where they are often palpated for “pulse”), characteristics of the carotid waveform can be readily recorded and analyzed using high fidelity pressure monitors placed over the skin of the neck 6. Importantly, the present invention exploits a derived physiological relationship that is not susceptible to data corruption from implantation of hardware in an aggressive biological environment. Waveform analysis is an established technique for studying cardiovascular characteristics, such as heart valve function, or atherosclerosis. However, to the best of Applicants' knowledge, it has never before been investigated as a method for measuring a derived physiological parameter such as ICP. The relationship between the carotid waveform and ICP has been modeled in silico and at the bench, prior to optimization and validation of the ICP algorithm in an animal model.

The concept of the present invention 20 for non-invasive determination of ICP is that features of the pulse pressure waveform in the arteries supplying the brain contain signals that are informative of the compliance and pressure in the cerebral vessels. These signals are detectable by a strategy known as blood pressure wave analysis. Blood pressure wave analysis (or pulse contour analysis) involves the evaluation of the shape of the arterial pressure wave over the course of one or more cardiac cycles. The idea that pressure waveforms encode qualitative and quantitative information about local or systemic hemodynamics is known. The behavior of pressure waves in arteries, and the pressure waveform, has previously been demonstrated to be dependent on the properties of the arterial tube, and on the system that terminates the arterial tube (Ref. 29). According to the Windkessel model and its modifications (Ref. 30), arterial blood pressure should increase and decay exponentially during each diastolic interval with a time that is determined by the peripheral resistance and the (nearly constant) arterial compliance. Because the pressure waveform incorporates these resistance and compliance factors, their analysis has been greatly explored as indicators of cardiovascular function, including cardiac output (Ref. 31), coronary heart disease (Ref. 32), evaluation of left ventricular assist device function (Ref. 33), and hypertensive pregnancy disorders (Ref. 34). However, over short time scales, peripheral arterial blood pressure waveforms are complicated, even dominated, by highly complex reflection waves propagating back and forth as blood moves through the ever narrowing branches of the arterial tree. These pressure wave reflections confound interpretations of arterial pressure waveforms for generalized cardiovascular function (e.g., for determining cardiac output), however they can be highly informative about local conditions of resistance and compliance (Ref. 35), and it is this feature that is exploited in the waveform analytical method of the present invention.

Of the main vessels that supply the brain, the two largest arise from the common carotid arteries 4 (FIG. 1A), the vessels in the neck 6 that are commonly palpated for arterial pulse. The carotid arterial pressure wave travels to the brain 1 where it is partially reflected upon striking the smaller diameter (and higher hydraulic impedance) cerebral vascular bed. This reflection contributes complexity to the overall shape of the carotid pressure waveform. As ICP increases in and around the brain, the cerebral vasculature is compressed, and the compliance of these vessels decreases. This reduction in cerebral vascular compliance leads to an increased impedance mismatch between the carotid arteries and the cerebral vessels. The extent of the impedance mismatch between the carotid arteries and cerebral vascular bed should be manifest by the strength of the components of the carotid pressure waveform that are contributed by pressure wave reflection (Ref. 36). The present invention 20 is capable of quantitatively interpreting these carotid pulse wave signals with respect to intracranial pressure.

As shown in FIG. 2A, the sensor portion 22 (and reference sensor 22a) are hand-held, each incorporating a highly sensitive pressure tonometer that can be placed on the skin overlying the palpable carotid artery (primary sensor 22) and overlying a reference artery (reference sensor 22a). The analyzer 24 also incorporates an analytical algorithm capable of qualitatively and quantitatively identifying informative signals from the pressure data being collected, and converting these into a value for ICP. Previous data demonstrate that reflected waves can be detected in the human cerebral circulation (Ref. 37). Furthermore, ICP has previously been correlated with the compliance characteristics of the jugular vein (Ref. 38). These data support the link between ICP and hemodynamic characteristics.

The present invention 20 utilizes arterial pulse pressure waveform analysis to derive intracranial pressure. Compared to existing methods of determining ICP, the present invention 20 is more rapid and easier to use, safer, possibly more accurate, and less expensive to produce and operate. It is entirely non-invasive, avoiding the inherent risks associated with surgery, such as anesthetic accident and infection. It is entirely portable, enabling repeat monitoring of ICP in an ambulatory setting (such as by first responders, or following discharge from the ICU). Furthermore, because the sensors 22/22a are not in direct contact with a biological tissue, there is no measurement drift or issues associated with calibration. Thus, in the method 100 (FIGS. 2B-2C) of the present invention 20, the ICP is not measured directly, but rather is derived from a related measurement, arterial pressure wave shape.

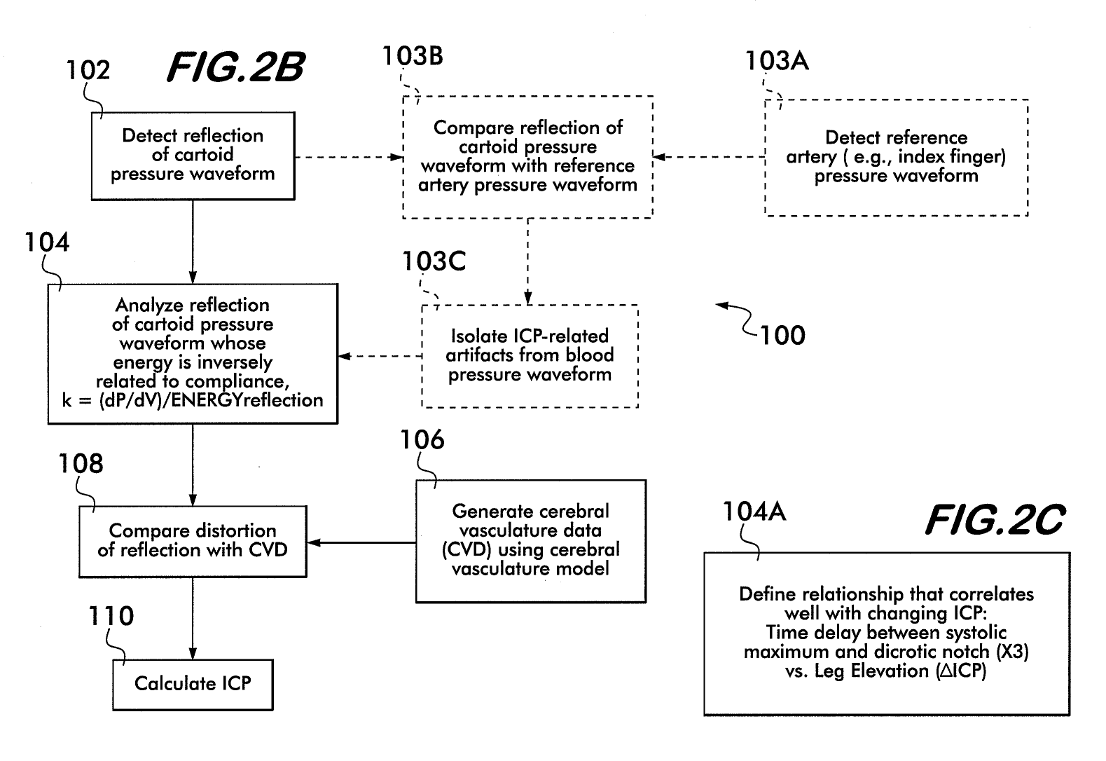

In implementing the present invention 20 which uses the carotid artery blood pressure waveform (CABPW) as a correlate for ICP, a high-fidelity system is used. There is a known non-linear, monotonic relationship between ICP and intracranial volume (see FIG. 3), the major determinant of intracranial compliance (dP/dV) and impedance (P(t)/F(t)). In particular, elevated ICP is linked to intracranial volume by this non-linear, monotonic relationship in the cranium. The carotid artery pulse increases both volume and pressure as it enters the cranium. Elevated intracranial volume results in higher intracranial compliance (dP/dV) and impedance ((P(t)/F(t)). In particular, when the carotid energy pulse enters the cranium, this increases the volume and pressure. The pressure wave reflected back from the cranium depends on cranium impedance. The energy of the reflected wave is inversely proportional to the compliance, and so, via this volume/pressure relationship, may be indicative of ICP. Thus, the lower the compliance (dP/dV), the more energy that will be reflected back toward the heart, i.e., modification of the carotid artery pressure waveform. By identifying parts of the waveform generated by the reflection, impedance changes linked to the ICP can be monitored.

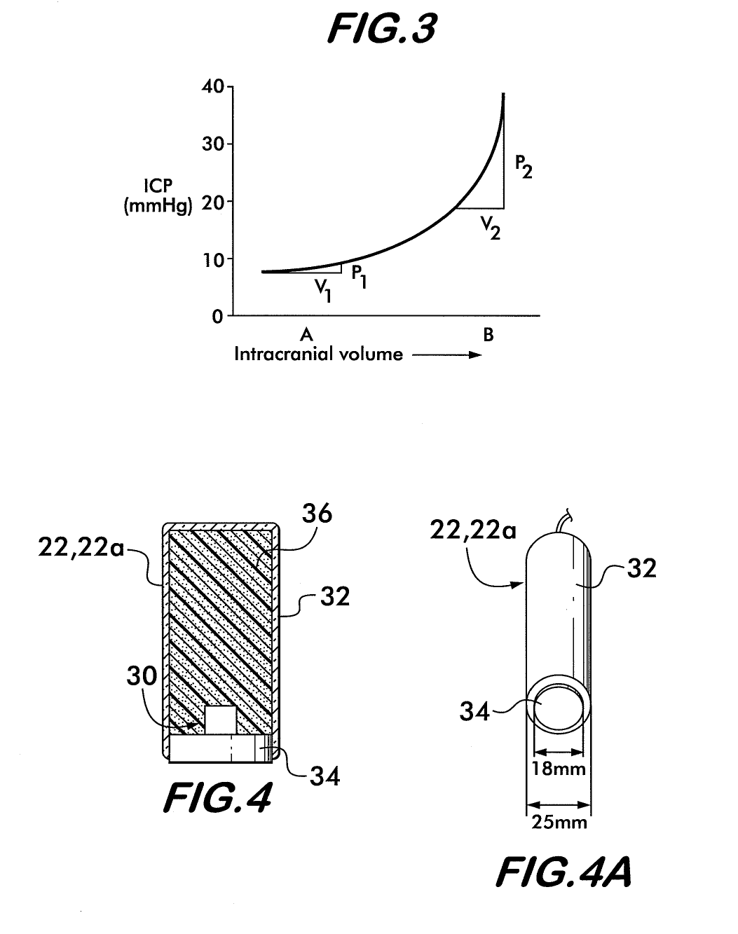

To investigate this, CABPW were analyzed in three healthy male volunteers, ages 25-40 (Refs. 25-40). As shown in FIGS. 4-4A, a custom-made arterial applanation tonometer was constructed (Ref. 39). A pressure die and associated electronics 30 includes a transducer which comprises a modified piezoresistive sensor (e.g., Freescale MPVZ5010) covered with a thin silicon film, and mounted inside an 18 mm (ID) acrylic tube 32 (FIGS. 4-4A). The specific geometry was selected to accommodate a wide range of external carotid artery anatomical characteristics and measurement conditions. The external rim 34 of the tube 32 facilitated stable conditions in the sensing area by mechanically stabilizing and shielding the piezoresistive sensor from motion artifacts, while a long handle (i.e., the tube 32) assisted the operator in optimizing the sensor angle to the subject's artery. In particular, the external rim 34 comprises, for example, a silicone pressure coupling and wherein the tube 32 itself is filled with, for example, an ultra-light filling foam 36. The voltage output from the transducer was connected to a data acquisition system (e.g., InstruNet®), and data were analyzed offline (the data acquisition and analysis scheme is shown in FIG. 4B).

As shown in FIG. 4B, the analyzer 24 comprises an instrumentation amplifier 38 an analog-to-digital converter (ADC) 40, digital filtering function 42, pulse detection algorithm function 44, pulse averaging function 46 and a parameter extraction function 48. As will be discussed in detail later, the output signal, a particular extracted parameter, designated “X3” was determined to correlate well with ICP. As can also be seen in FIG. 4B, the piezoresistive transducer detects the CABPW which has the pulse form shown and forms the input signal to the present invention 20.

CABPW was measured in subjects in which ICP was modified by elevating the legs, a method that is similar to using a tilt table (Ref. 40). Five elevations were tested (0, 14, 28, 42, 68 cm), with a 10 minute equalization period between each elevation to ensure stable cranial pressure conditions. For each elevation, several minutes of pulse trains were collected at a sampling frequency of, for example, 1000 Hz. The frequency response of the device was tailored to measure all high harmonics present in the signal, with a high signal to noise ratio. No attempt was made to introduce a calibration procedure to the system, since the dynamic range is relatively constant, and correlating to the absolute arterial pressure was outside the scope of this preliminary experiment.

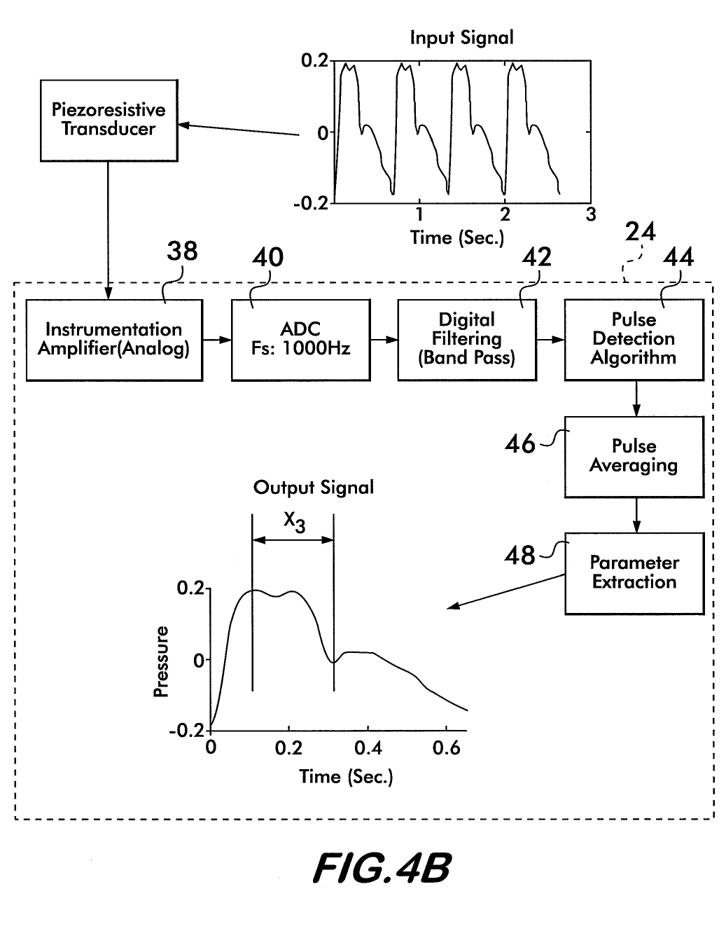

Data were analyzed offline. The signal was filtered and the DC component (signal offset represented in Fourier series by coefficient a₀) was eliminated, so only the dynamic components of the signal remained (represented by coefficients a₁, a₂ . . . ; b₁, b₂ . . . etc.). A “typical” representative pulse at each leg elevation was constructed from a train of pulses collected during the experiment (FIG. 5A). This representative pulse contained “averaged” features of the CABPW normalized for cycle duration, and was used to mine for ICP-dependent features. Cycles were extracted from the signal and analyzed using custom software. The representative pulse wave was analyzed in time, frequency, and wavelet domains, as well as on the phase plane.

A strong, highly linear relationship (r²=0.98; 0.88; and 0.66 for three subjects) was identified between leg elevation and at least one key characteristic of the CABPW: the time delay between the systolic maximum and the dicrotic notch, a parameter designated as X3, FIG. 5A. The relatively high consistency between three subjects (FIG. 5B) suggests that the relationship between X3 and leg elevation (a surrogate for ICP that may require independent verification in future studies) is subject-independent. Phase plane, wavelet, and frequency analyses also exhibited promising, though less consistent, relationships with leg elevation. These data demonstrate that a simple, high-fidelity data collection system that requires no calibration, and a novel data analysis technique, can be used to differentiate levels of leg elevation in three subjects. Further development and optimization of the signal collection and analysis methods, could improve the consistency of the algorithm, leading to a robust, non-invasive, simple, and inexpensive ICP (cranial impedance) monitor.

As mentioned previously, a reference sensor 22a can be used in the system 20 and method 100 of the present invention. In particular, the reference sensor 22a is used to collect a reference pulse (e.g., also using a tonometer) on the radial artery or index finger (FIG. 2A), or any other artery remote from the carotid artery.

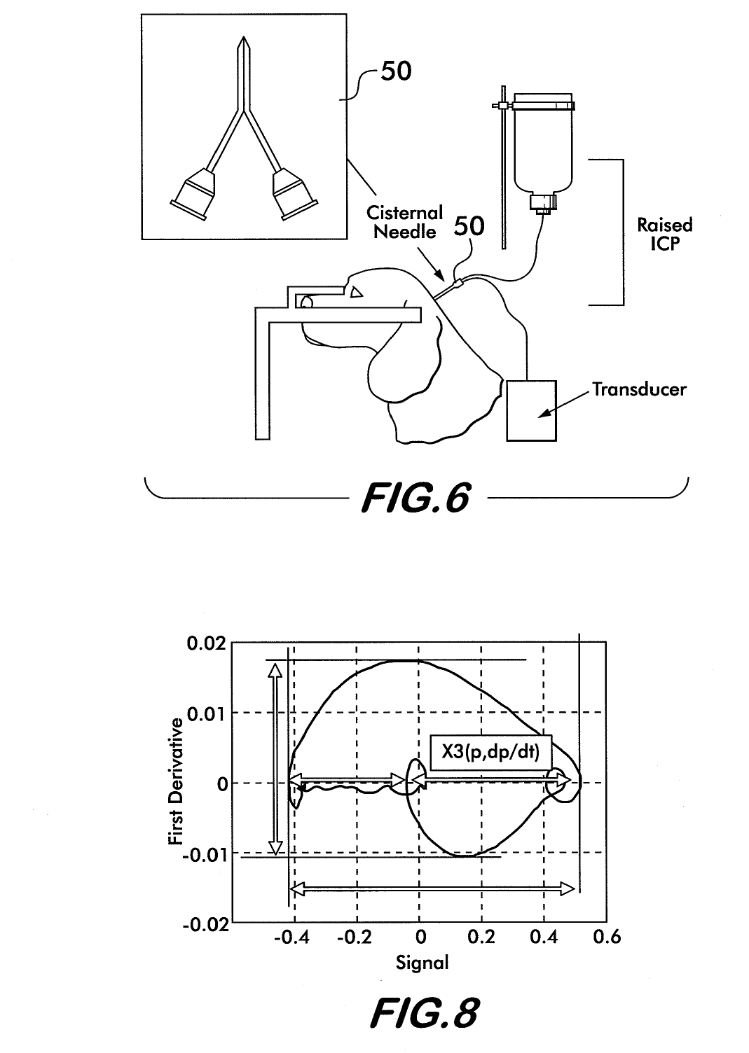

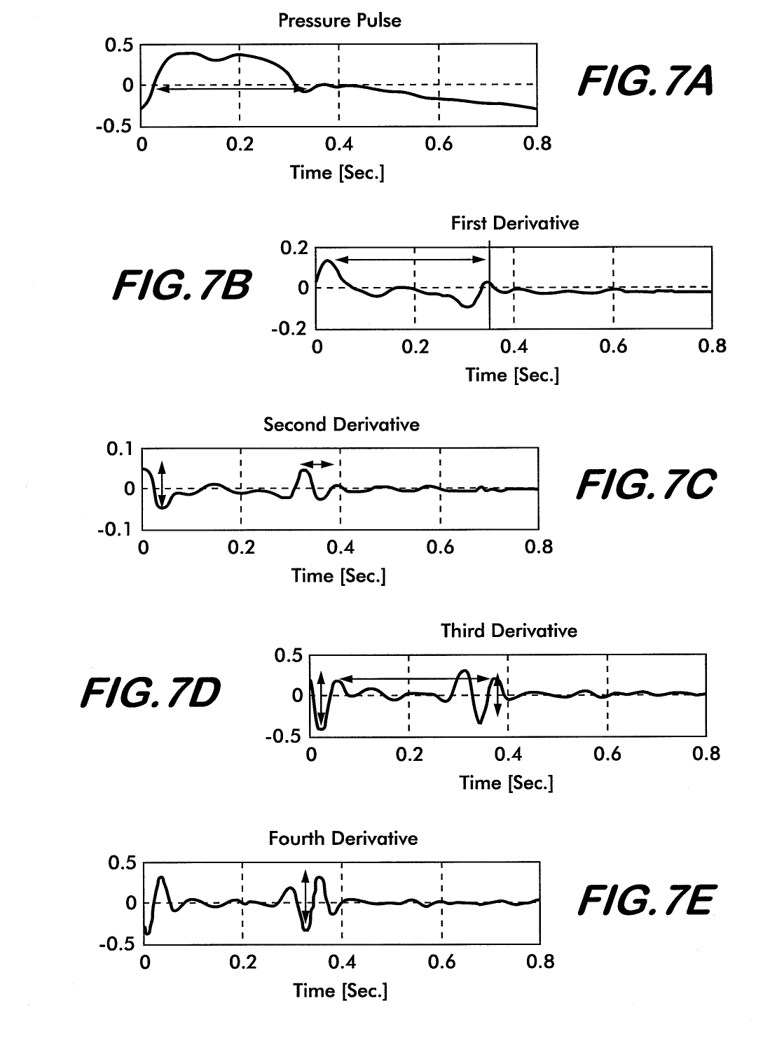

An alternative is to combine an optical plethysmography (a reference signal recorded on the index finger) with the external carotid artery waveforms. The detection of the reference pulse facilitates compensating for changes caused by the systematic impedance. Two measurement sites separated by a long artery provides information related to different sections of the circulatory system. As a result, the phenomena caused by ICP or intracranial volume (ICV) are more apparent if compared to a reference signal. The parameters measured are time differences between maxima/minima of the signals (carotid and reference) and subsequent derivatives shown in FIGS. 7A-7E. Other approaches include analysis of derivatives which can help in finding characteristic points of the signal (e.g., as those shown in FIGS. 7A-7E, which show typical series of pulse and its derivatives). These methods are similar to “acceleration plethysmography” but instead of using an optical signal, this uses pressure waveform collected on the carotid arteries. FIG. 8 also depicts a phase-plane analysis (CABPW vs. first derivative in time) depicting the parameter X3, as well as other parameters. These changes are correlated to the ICP.

The present invention 20 and method 100 includes two objectives:

Objective 1: Develop an algorithm for determining ICP from features of the carotid waveform. Validated mathematical and bench models of the cerebral vasculature have been used to investigate the pulse pressure waveform in the carotid arteries under different values of simulated ICP. Features of the waveform that vary monotonically with ICP are identified and used to develop an algorithm for determining ICP.

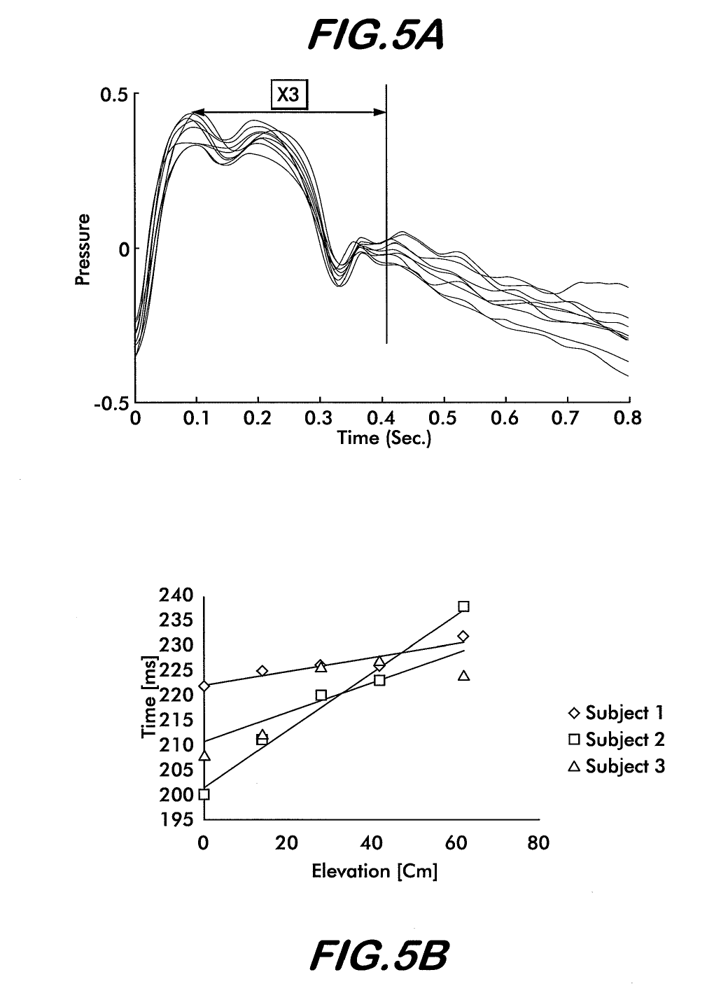

Objective 2: Optimize and validate the carotid waveform algorithm in an animal model of cerebral hypertension (see FIG. 6). The algorithm may be optimized using an animal model in which a carotid pulse waveform is monitored while ICP is varied. Once the algorithm has been optimized, it is validated in a blinded experiment in which ICP measured using the pulse wave traces are then correlated to an independent “gold standard” measure of ICP.

Effectiveness of the pulse waveform analysis method for determining ICP may be demonstrated, if at least one feature of the carotid waveform exhibits a quantitative monotonic relationship with ICP (r²>0.9). The hypothesis is that a monotonic relationship exists between ICP and one or more quantitative features of the pulse pressure waveform in the common carotid artery. This concept is developed in an in vitro model of brain vasculature and cerebrospinal fluid. Cardiovascular flow models have been in development for more than 40 years and have become highly sophisticated. They enable flow variables to be studied, and promising patterns to be identified prior to validation in animals and humans. These models are ideal for early testing of conceptual hypotheses of biological fluid dynamics. Well-described strategies are then adapted for modeling fluid flow through a vascular system to the unique situation of the cerebral vasculature, where compliant brain and vascular structures are encased within a rigid environment imposed by the skull. A mathematical model is used to investigate the behavior of the cerebral hydrodynamic system and to guide development of an algorithm for determining ICP from the carotid pulse waveform using a bench mock circulation model (Objective 1). Once informative signals within the carotid waveform have been identified, and their relationship to ICP predicted, the algorithm is then optimized in an animal model (FIG. 6) in which cerebral vascular tension and ICP can be controlled. Following optimization, the strategy can be validated in a blinded experiment (Objective 2)

Objective 1: Develop an Algorithm for Determining ICP from Features of the Carotid Waveform

In Objective 1, design inputs for ICP measurement by arterial waveform analysis are determined by modeling the test system. The Windkessel strategy and transmission line theory are well-described and commonly-used mathematical methods for modeling cardiovascular systems (Ref. 39). Here these are adapted to describe the hydrodynamic relationship between the cerebral arterial supply, capillary and CSF fluid reservoirs, and the venous drainage from the brain. The model is built on an anatomical “map” of the vasculature of the head and brain, starting from the common carotid arteries, and ending with the jugular veins. Each anatomical element (e.g. artery, arteriole, venule) within the system is assigned fixed values, derived from the literature, for compliance and resistance, reflecting the diameters and viscoelastic properties of each vessel (Ref. 41). The CSF, the rigid enclosure of the skull, the elastic properties of the brain tissue, and the compressible vascular bed of the brain are modeled as unique modifying features of the system. The Windkessel and transmission line theory models describe the pulsatile flow behavior of blood (including complex reactive and reflective pressure wave characteristics at impedance interfaces) within each element of a system, when input and output, and modifying factors, are varied. In the present invention 20 and method 100, these variable factors include pressure and flow characteristics of waveforms entering the system via the common carotid arteries (input), the volume of blood in the venous system (output), and (importantly) the compression of vessels and capillaries of the brain by pressure exerted from the surrounding CSF (modifier). It should be noted that pressure (P) and flow (F) are calculated as periodic functions of time: P(t+T), F(t+T), where T is the heartbeat period calculated from the heart rate (HR=1/T). Previous studies have modeled the relationship between ICP and extracranial arterial blood flow (c.f. pressure) measured by Doppler (Ref. 42), providing important brain fluid dynamic models that are used to inform the present invention 20. By way of example only, Matlab® software is used to simulate pressure an flow conditions through the model system, and to monitor the carotid pulse waveform as each factor of interest is varied. Matlab® is a numerical computing environment that more readily enables interpretation and manipulation of complex matrices than other software languages such as C++, Visual C, or Visual Basic. Features of the carotid waveform are analyzed for dependence on the ICP (as discussed in detail below), and these signals form the basis for a hypothesized predictive algorithm of ICP. A physical bench model may be used to test these hypothesized relationships.

A mock circulatory model of the cerebral vasculature may be used to test the carotid waveform-ICP concept. The major vessels of the head and brain, starting with the common carotid arteries, and ending with the jugular veins, can be modeled using silicon tubing. Silicon “vessels” can be obtained commercially (e.g., Dynatek) in a wide variety of thicknesses and diameters, closely mimicking the viscoelastic properties of diverse types of blood vessels. The arborization of the head and brain vasculature are modeled down to vessels with a diameter of 1 mm, and contain a fluid (water and glycerol) with the same viscosity as blood. The effects of arterial branches that leave the system (e.g. the external carotid) are then mimicked using a hydraulic resistor (e.g., a valve) that recreates the cumulative compliance of the exiting arteries and their branches. The smallest of the vessels within the system (e.g. the cerebral arterioles and capillary bed) is then collectively simulated using a compressible hydraulic resistance “bed”, housed within a rigid box (to simulate the skull) containing mock CSF fluid. The volume and pressure of this mock CSF are adjustable. The surrogate blood is then pumped through the model using a commercial blood pressure calibration pump (a pulse duplicator that mimics the input of flow/pressure waveforms, e.g., Dynatek). Pressure waveforms in the common carotid element of the system are monitored using a standard pressure transducer and flow wave monitored using an electromagnetic flow probe. Data is analyzed using, by way of example only, an InstruNet® model 100 HC data collection and analysis system. The effect on the carotid waveform of different “CSF” pressures on the compressible cerebral vasculature are measurable at different carotid input flow rates and venous output resistances. These measurements are compared to those determined in the mathematical model. The mathematical and bench models therefore are refined in an iterative manner. At the end of this objective, one or more features of the common carotid waveform are identified that the mathematical model and the bench model both indicate is/are dependent on the ICP. These features are used in an algorithm for predicting ICP, to be validated using an animal model.

### Objective 2: Optimize and Validate the Carotid Waveform Algorithm in an Animal Model of Cerebral Hypertension.

Although simulations and inert models provide the ease and rapidity with which large numbers of developmental tests can be performed to understand the general behavior of a system, they cannot capture the complexity of a living organism. Here an animal testing model is used to optimize and validate the ICP measurement algorithm developed using the mathematical and bench models. An animal model is adapted from one that has previously been utilized for manipulating ICP (Ref. 43). For this experiment, a large mammal is needed to simulate human cervical vascular anatomy. For best results, a tractable but non-companion animal, e.g., a sheep, is best suited for this purpose. All procedures will be carried out in accordance with policies set forth by the local Institutional Animal Care and Use Committee and in accordance with NIH guidelines for the humane and ethical treatment of animals.

The waveform method for ICP measurement determined using the models in Objective 1 is optimized by recording carotid pulse pressure waves over a wide range of ICP levels, and adjusting the algorithm incrementally. Briefly, the sheep is anesthetized, placed in a stereotaxic head holder, and prepared for surgery. A double-bored needle 50 (FIG. 6) is inserted into the cisterna magna. (The cisterna magna is a subarachnoid cavernous space, between the cerebellum and the medulla oblongata, into which CSF drains. It is spatially continuous with the brain ventricles). One branch of the needle is connected to a pressure transducer and the other branch, via flexible tubing, is connected to a reservoir bottle containing mock CSF. This enables ICP to be measured as an independent “gold standard”, and also facilitates the infusion of mock CSF into the ventricles to produce a range of ICP. During each experimental session, ICP is adjusted by raising the reservoir to an empirically-determined height above the animal until ICP stabilizes, as determined from cisternal pressure recording. The carotid pulse waveform is monitored simultaneously using a skin pressure transducer and electromagnetic flow probe. At the end of each experimental session, ICP is allowed to return to normal, the cisternal needle is removed, and the animal is allowed to awaken.

Once the waveform algorithm has been optimized, the design specifications are “locked”, and the ability to accurately determine ICP from carotid pulse waveform analysis is tested in a blinded trial. The same animal model is utilized. Cisternal ICP and carotid pulse waves are recorded at a range of ICP levels, from 5-60 mm Hg, and in a random, pre-determined order. ICP is interpreted from the pulse waves, using the optimized algorithm, by an investigator blinded to cisternal ICP readings. After all recordings have been made, the animal is euthanized, and the carotid pulse waveform ICP values correlated to the cisternal ICP readings.

As mentioned previously, in each model (mathematical, bench, animal), the common carotid waveform is continuously monitored at a variety of CSF pressures. The relationship between various quantitative features of the carotid waveform and the ICP are investigated graphically (by plotting how each feature varies with CSF pressure). Examples of waveform features include wave amplitude, wave systolic-diastolic gradients, ratios of harmonics after Fourier analysis, times between waveform features, distances between waveform features on the phase plane, area of the cycles on the phase plane, power of the reflected waveform, and amplitude of the reflected waveform. It is expected that a number of waveform features may show a relationship with CSF pressure. Those that are common (at least qualitatively) to all models, or that show strong relationships in one or more of the models, are tested in a predictive algorithm of ICP. This algorithm is validated using carotid pulse waveform traces collected from the sheep model, and analyzed blindly. As mentioned previously, the effectiveness of the pulse waveform analysis method for determining ICP is demonstrated, if at least one feature of the carotid waveform exhibits a quantitative monotonic relationship with ICP (r²>0.9).

The major risk is that the models do not reproduce physiological carotid artery waveforms for some ICP levels, and that the resulting algorithm fails in vivo. A possible alternative would be to use an active impedance module which, instead of being a passive resistor/capacitor element, is an active pulse duplicator which adjusts parameters in real time to obtain desired waveforms (closed loop system which adjusts resistance and capacitance based on sensor input). Although the use of bench models can accelerate the development of an informative algorithm, if they fail, a “generic algorithm” can be developed to define model parameters, using carotid artery waveforms measured in vivo (in Objective 2). The generic algorithm method is an “intelligent iteration” using an initial combination of parameters that are adjusted toward the target.

In vivo measurement: Despite promising preliminary data, it is possible that the proposed method for deriving ICP may be too strongly influenced by other parameters of the circulatory system, producing unreliable results. In this case, as mentioned previously, a reference pulse is utilized, collected at a “control” artery (such as the radial artery pulse, or the finger pulse), to compensate for systemic impedance. Two measurement sites, separated anatomically, provide information related to different sections of the circulatory system allowing phenomena related to ICP and intracranial volume to be more readily identified. Other strategies include analyzing pulse derivatives (examples are provided in FIGS. 7A-7E), which can exhibit characteristics not identifiable in non-derivized data. This method is similar to “acceleration plethysmography”, but using the carotid artery pressure waveform instead of using an optical signal. Optical plethysmography, using a reference signal recorded on the index finger, for example, could also be combined with the carotid artery blood pressure waveform analysis.

In view of the foregoing, there is shown in FIGS. 2B-2C a flow diagram of the method 100 of the present invention. In step 102, using the primary sensor 22 placed over the carotid artery of the living being, a reflection of the carotid pressure waveform is detected. In step 104, the analyzer 24 analyzes the reflection based on the relationship that the intracranial compliance (dP/dV) and the energy of the reflection are inversely related manifested through the wave distortion. In step 106, which can occur prior to any of these steps, or be done concurrently, cerebral vasculature data (CVD) is generated using cerebral vascular model(s). In step 108, the wave distortion data is compared with the CVD. In step 110, the ICP is determined from the comparison conducted in step 108. As also discussed previously, one well correlated parameter, X3, obtained through the analysis of the reflection of the carotid pressure waveform is the time delay between the systolic maximum and the dicrotic notch versus the living being's leg elevation. Therefore, an exemplary implementation of step 104 is provided by step 104C as shown in FIG. 2C.

As mentioned previously, it is preferable, but not required, to include a reference sensor 22a that detects a reference artery pressure waveform remote from the carotid pressure waveform, e.g., the artery in the index finger. If a reference sensor 22a is used, the method 100 is modified to include steps 103A-103C. In particular, in step 103A the reference sensor 22a is used to detect a reference pressure waveform. In step 103B, this reference pressure waveform is compared to the detected carotid pressure waveform of step 102. In 103C, ICP-related artifacts are isolated from this comparison of the two pressure waveforms.

### REFERENCES

1. Steiner, L. and P. Andrews, Monitoring the injured brain: ICP and CBF. Br J Anaesthesia, 2006. 97: p. 26-38.
2. Czosnyka, M., et al., Vascular components of cerebrospinal fluid compensation. J Neurosurg, 1999. 90: p. 752-759.
3. Dayson, H., G. Hollingsworth, and M. Segal, The mechanism of drainage of the cerebrospinal fluid. Brain, 1970. 93: p. 665-678.
4. Marmarou, A., et al., Contribution of CSF and vascular factors to elevation of ICP in severely head-injured patients. J Neurosurg, 1987. 66: p. 883-890.
5. Miller, J., A. Stanek, and T. Langfitt, Concepts of cerebral perfusion pressure and vascular compression during intracranial hypertension. Prog Brain Res, 1972. 35: p. 411-432.
6. Bullock, R., Mannitol and other diuretics in severe neurotrauma. New Horizons, 1995. 3:p. 448-452.
7. Patel, H., et al., Specialist neurocritical care and outcome from head injury. Intensive Care Med, 2002. 28: p. 547-553.
8. Miller, J., et al., Further experience in the management of severe head injury. J Neurosurg, 1981. 54: p. 289-299.
9. Marshall, L., R. Smith, and H. Shapiro, The outcome with aggressive treatment in severe head injuries. Part I: the significance of intracranial pressure monitoring. J Neurosurg, 1979. 50: p. 20-25.
10. Patel, H., et al., Mortality after head trauma. Effect of neurosurgical care. Acra Neurochir (Wien), 2003. 145: p. 1135-1148.
11. Saul, T. and T. Ducker, Effect of itnracranial pressure monitoring and aggressive treatment on mortality in severe head injury. J Neurosurg, 1982. 56: p. 498-503.
12. Tuite, G., et al., The beaten copper cranium: a correlation between intracranial pressure, cranial radiographs, and computed tomographic scans in children with craniosyntosis. Neurosurgery, 1996. 39: p. 691-699.
13. Sussman, J., N. Sarkies, and J. Pickard, Benign intracranial hypertension. Pseudotumour cerebri: idiopathic intracranial hypertension. Adv Tech Stand Neurosurg, 1998. 24: p. 261-305.
14. Tissell, M., et al., Elastance correlates with outcome after endoscopic third ventriculostomy in adults with hydrocephalus caused by aqueductal stenosis. Neurosurgery, 2002. 50: p. 70-77.
15. Rebaud, P., et al., Intracranial pressure in childhood central nervous system infections. Intensive Care Med, 1988. 14: p. 522-525.
16. Schwab, S., et al., The value of intracranial pressure monitoring in acute hemispheric stroke. Neurology, 1996. 47: p. 393-398.
17. Mayer, S. and J. Chong, Critical care management of increased intracranial pressure. J Int Care Med, 2002. 17: p. 55.
18. Gambardella, G., D. d'Avella, and F. Tomasello, Monitoring of brain tissue pressure with a fiberoptic device. Neurosurgery, 1992. 31: p. 918-922.
19. Miller, J. and e. al., Inaccurate pressure readings for subarachnoid bolts. Neurosurgery, 1986. 19: p. 253-255.
20. Barth, K., et al., A simple and reliable technique to monitor intracranial pressure in the rat: technical note. Neurosurgery, 1992. 30: p. 138-140.
21. Czarnik, T., et al., Noninvasive measurement of intracranial pressure: is it possible? JTrauma, 2007. 62: p. 207-211.
22. Glick, R., et al., Early experience from the application of a noninvasive magnetic resonance imaging-based measurement of intracranial pressure in hydrocephalus. Neurosurgery, 2006. 59: p. 1052-1060 (discussion 1060-1061).
23. Voss, S., et al., Posture-induced changes in distortion-product otoacoustic emissions and the potential for noninvasive monitoring of changes in intracranial pressure. Neurocrit Care, 2006. 4: p. 251-257.
24. Zhao, Y., J. Zhou, and G. Zhu, Clinical experience with the noninvasive ICP monitoring system. Acta Neurochir, 2005. 95 Suppl: p. 351-355.
25. Schmidt B, B. S., Pässler, M., et al., Fuzzy pattern classification of hemodynamic data can be used to determine noninvasive intracranial pressure. Acta Neurochir, 2005. 95 Suppl: p. 345-349.
26. Dutton, R., et al., Preliminary trial of a noninvasive brain acoustic monitor in trauma patients with severe closed head injury. J Trauma, 2002. 53: p. 857-863.
27. Michaeli, D. and Z. Rappaport, Tissue resonance analysis; a novel method for noninvasive monitoring of intracranial pressure. J Neurosurg, 2002. 96: p. 1132-1137.
28. Ueno, T., et al., Development of a noninvasive technique for the measurement of intracranial pressure. Biol Sci Space, 1998. 12: p. 270-271.
29. Sugimachi, M., et al. Estimation of arterial mechanical properties form aortic and tonometric arterial pressure waveforms. in Working Conference on Biosignal Interpretation (BSI). 1997.
30. Sipkema, P., N. Westerhof, and O. Randall, The arterial system charachterized in the time domain. Cardiovasc Res, 1980. 14: p. 270-279.
31. Linton, N. and R. Linton, Estimation of changes in cardiac output from the arterial blood pressure waveform in the upper limb. Br J Anaesthesia, 2001. 86: p. 486-496.
32. Nishijima, T. and e. al., Pulsatility of ascending aortic blood pressure waveforms is associated with increased risk of coronary heart disease. Am J Hypertens, 2001. 14: p. 469-473. 33. Kawahito, S., et al., Analysis of the arterial blood pressure waveform during left ventricular nonpulsatile assistance in animal models. Artificial Organs, 2000. 24: p. 816-820.
34. Faber, R., et al., Analysis of blood pressure waveform: a new method for the classification of hypertensive pregnancy disorders. J Human Hypertension, 2004. 18: p. 135-137
35. Noodergraaf, A., Circulatory System Dynamics. 1978, New York: Academic.
36. Nichols, W., McDonald's Blood Flow in Arteries: Theoretical, Experimental and Clinical Principles. 2005, Hodder Arnold: London. p. 199-200.
37. Diehl, R., Wave reflection analysis of the human cerebral circulation during syncope. Auton Neurosci, 2007. 132: p. 63-69.
38. Cirovic, S., C. Walsh, and W. Fraser, Mathematical study of the role of non-linear venous compliance in the cranial volume-pressure test. Med Biol Eng Comput, 2003. 41: p. 579-588.
39. Nichols, W., McDonald's Blood Flow in Arteries: Theoretical, Experimental and Clinical Principles. 2005, Hodder Arnold: London. p. 466-467.
40. Manwaring, P., et al., A signal analysis algorithm for determining brain compliance noninvasively. Eng Med Biol Soc, 2004. 1: p. 353-356.
41. MCDonald, D., The relation of pulsatile pressure to flow in arteries. J Physiol, 1955. 127:p. 533-552.
42. Penson, R. and R. Allen. Simulation and validation of intracranial pressure. in Proceedings of the 10th European Simulation Symposium. 1998. Nottingham.
43. Marshall, L., et al., Effects of severe experimental intracranial hypertension on the rabbit brain. Surg Forum, 1974. 25: p. 456-458.

While the invention has been described in detail and with reference to specific examples thereof, it will be apparent to one skilled in the art that various changes and modifications can be made therein without departing from the spirit and scope thereof.

1. A system for measuring intracranial pressure (ICP) of a living being non-invasively, said system comprising:
a sensor for detecting blood pressure non-invasively;
an analyzer that receives the blood pressure information and derives at least one parameter that correlates with ICP to provide ICP data from the blood pressure information; and
an output device for displaying said ICP data.

2. The system of claim 1 wherein said sensor comprises a pressure sensor for non-invasively detecting a blood pressure waveform related to carotid artery blood pressure.

3. The system of claim 2 wherein said blood pressure waveform comprises a feature of a carotid artery blood pressure waveform (CABPW).

4. The system of claim 3 wherein said feature of said CABPW exhibits a quantitative monotonic relationship with ICP such that r²>0.9, wherein r represents a correlation coefficient.

5. The system of claim 4 wherein said at least one parameter comprises a time delay between systolic maximum and the dicrotic notch.

6. The system of claim 3 wherein said analyzer comprises time analyses, frequency analyses and wavelet domain analyses.

7. The system of claim 6 wherein said analyzer comprises analog to digital conversion, digital filtering, pressure pulse detection, pulse averaging and parameter extraction.

8. The system of claim 7 wherein said analyzer evaluates a plurality of time derivatives of said feature of said CABPW.

9. The system of claim 1 wherein said sensor comprises high fidelity pressure monitor.

10. The system of claim 9 wherein high fidelity pressure monitors comprise pressure tonometers.

11. The system of claim 10 wherein said pressure tonometers comprise piezoresistive transducers.

12. The system of claim 11 wherein said sensor comprises an external rim that contacts the skin of the living being while stabilizing and shielding said piezoresistive transducer from motion artifacts.

13. The system of claim 2 wherein said system comprises a second sensor, coupled to said analyzer, for detecting a blood pressure non-invasively and compensating for blood system impedance, said reference blood pressure being located remotely from the carotid artery blood pressure.

14. The system of claim 13 wherein said second sensor comprises a high-fidelity pressure monitor.

15. The system of claim 14 wherein said high fidelity pressure monitor comprises a piezoresistive transducer.

16. The system of claim 1 wherein said output device comprises a monitor.

17. A method for measuring intracranial pressure (ICP) of a living being non-invasively, said method comprising:
non-invasively detecting blood pressure of the living being;
analyzing a feature of said detected blood pressure that correlates with ICP to provide ICP data from said feature of said detected blood pressure;
calculating ICP from said feature of said detected blood pressure.

18. The method of claim 17 wherein said step of non-invasively detecting blood pressure comprises non-invasively detecting a carotid blood pressure waveform.

19. The method claim 18 wherein said step of analyzing a feature of said detected blood pressure comprises deriving an impedance mismatch between carotid arteries and cerebral vessels via a reflection of the carotid pressure waveform.

20. The method of claim 19 wherein said step of calculating ICP from said feature of said detected blood pressure information comprises calculating ICP from said impedance mismatch and said reflection.

21. The method of claim 19 wherein said step of analyzing a feature comprises detecting a feature of a carotid blood pressure waveform (CABPW) that exhibits a quantitative monotonic relationship with ICP such that r²>0.9, wherein r represents a correlation coefficient.

22. The method of claim 21 wherein said step of detecting a feature comprises a time delay between systolic maximum and the dicrotic notch.

23. The method of claim 19 wherein said step of analyzing a feature of said detected blood pressure comprises analyzing a reflection of a carotid blood pressure waveform (CABPW) whose energy is inversely related to intracranial compliance.

24. The method of claim 23 wherein step of analyzing said reflection comprises comparing the distortion of said reflection with known cerebral vasculature data generated using cerebral vasculature models.

25. The method of claim 23 further comprising the step of detecting a reference blood pressure, remotely-located from said carotid arteries, said reference blood pressure being compared with said CABPW to compensate for blood system impedance before analyzing said reflection of said CABPW.

26. The method of claim 18 wherein said step of non-invasively detecting blood pressure comprises positioning a pressure tonometer on the skin of the living being overlying the palpable carotid artery.

27. The method of claim 23 wherein said step of analyzing a feature of said detected blood pressure comprises analyzing said reflection of the carotid pressure waveform in time, frequency and wavelet domains.

28. The method of claim 27 wherein said step of analyzing said reflection of the carotid pressure waveform in time comprises evaluating a plurality of time derivatives of said CABPW.
